(19) United States
(12) Patent Application Publication
(10) Pub. No.: US 2013/0013036 A1
(43) Pub. Date: Jan. 10, 2013

(54) Aromatherapy Thermal Pad

(76) Inventor: Jacqueline Zaragosa, Salt Lake City, UT (US)

(21) Appl. No.: 13/177,641

(22) Filed: Jul. 7, 2011

(51) Int. Cl. A61F 7/00

(52) U.S. Cl. 607/112; 607/114

(57) ABSTRACT

An aromatherapy heating and cooling pad device is provided for the purpose of treating variety of aches, pains and injuries. The device comprises an aroma-permeable outer shell, a padded inner layer, an herbal composition and a securing means. The outer shell enclosed an all-natural padded inner layer that offers thermal insulation and comfortable protection to an injured area of a user. Stitching along the length of the device secures the outer shell to the inner layer and separates the inner space into chambers. These chambers are filled with an herbal composition of aromatic herbs and seeds that provide aromatherapy benefits to a user of the device. The device may be fastened to the affected area of a user by securing means attached at laterally opposing sides of the outer shell.

### CROSS REFERENCE TO RELATED APPLICATION

This application claims the benefit of U.S. Provisional Application No. 61/274,088 filed on Aug. 16, 2010, entitled “Naturally Basic.”

### BACKGROUND OF THE INVENTION

1. Field of the Invention

The present invention relates to therapeutic pain pad devices, and more specifically to an all-natural aromatherapy pain treatment pad.

In their everyday lives people often incur minor bodily injuries. Common actions such as walking, sitting, and eating can result in bumps, bruises, muscle pulls and strains. Intense activities can lead to overexertion of muscles and overextension of bones and ligaments. Other bodily aches and pains can surface due to exertion over a period of time causing extended periods of discomfort. Heat therapy is commonly is used to loosen sore or overextended muscles and ligaments. Placement of heat on sore or tense spots on the body can cause the underlying tissue to soften and relax, thus relieving pain of a user.

Heat packs or pads may be utilized to provide relief to a variety of injuries. These heat treatments may be electric heating pads, chemical gel packs, a hot moist towel, or other producer of thermal energy. Unfortunately these treatments are not adapted to remain in place without additional support. Injured persons often tie the thermal therapy pack to their injured limb in an effort to maintain the desired care on the area. This technique is problematic because the securing means is not connected to the therapy device and is thus unstable. Electric heating pads pose further limitation issues because they must be plugged in to a wall, preventing a user from becoming mobile or otherwise ambulatory.

Additionally, common heat therapy options are not environmentally friendly or biodegradable. Some use chemical gels to generate heat that can be harmful to a user and the environment if the pack is opened. The instant invention provides an all-natural and biodegradable heating pad having aromatherapy benefits. The construction materials are environmentally friendly and the aromatherapy benefits are provided by a quantity of herbs and seeds that are intrinsically biodegradable and therapeutic. The device offers attached securing means to allow a user mobility while the device is in use. To apply heat to the pad, it may be placed in an oven or microwave for quickly raising the temperature of the pack contents. Conversely, if the pack is desired to ice certain wounds, the pack may be placed in the freezer for cooling thereof. It is therefore desired to provide an environmentally friendly aromatherapy-thermal pad that is modularly securable to fulfill a long felt in need in the art.

2. Description of the Prior Art

The prior art contains a variety of aromatherapy based devices and methods for treating aches and pains in various parts of a user's body. These devices have familiar design and structural elements for the purposes of treating musculoskeletal pain; however they are not adapted for the task of providing an all-natural aromatherapy heating pad that is modular and securable to a variety of locations along a user's body.

Mizrahi, U.S. Patent Publication No. 2008/0039913 discloses an aromatherapy pain treatment blanket having an aroma-permeable covering, subspaces housed within the same, said subspaces containing aromatherapy material and thermal reservoir material. The thermal reservoir material may consist of a variety of grains, beans, and/or flaxseed. The aromatherapy material may consist of a variety of herbs. A user desiring pain treatment may heat the blanket device and place it over the affected area. Mizrahi does not disclose a cotton therapy pad or a means of securing the device to a user's injured area after heating.

Unrug, U.S. Pat. No. 5,476,492 discloses a method for body warming by placing on the body elastic, breathable shell housing internal compartments said internal compartments being filled with whole seed rubefacient herbs. The shell may have varied shapes according to the intend area of application. To secure the device to the area closed elastic loops may be attached at opposing ends of the shell. The aromatherapy body warmer of Unrug does not disclose a cotton pad that may be tied to an injured area.

Mizrahi, U.S. Pat. No. 7,125,147 discloses an aromatherapy heating blanket for the neck and shoulders. Said blanket having and aroma permeable outer covering, a plurality of segregated compartments, said compartments housing thermal reservoir material and aromatherapy materials. The thermal reservoir material may consist of a variety of grains, beans, and/or flaxseed. The aromatherapy material may consist of a variety of herbs. An “X” shaped cutout is disposed in the center of the device to allow a user to place the device around his or her neck. In one embodiment, the device may have an opening extending from the center cutout to an outer edge of the device to provide or to facilitate ease of applying the device to a user's neck or shoulders. A first side of the opening is removably secured to a second opposing side of the same by a fastening means. Mizrahi does not disclose a cotton heating pad that may be secured to an injured area of a user.

Frank, U.S. Pat. No. 7,923,038 discloses a method of relieving pain by applying a topical treatment to the skin over the affected area. Said topical treatment comprising primarily herbal extracts emulsified in a water based gel. The herbal treatment method of Frank does not disclose the use of a cotton heated pad on the affected area of a user, nor does it describe a means for securing such a device.

The devices and methods disclosed by the prior art do not address the need for a securable and modular aromatherapy thermal pad. The current invention relates to a device for providing all natural thermal pain relief to a variety of locations on a user's body. It substantially diverges in structural elements from the prior art; consequently it is clear that there is a need in the art for an improvement to the existing aromatherapy thermal pad devices. In this regard the instant invention substantially fulfills these needs.

### SUMMARY OF THE INVENTION

In view of the foregoing disadvantages inherent in the known types of aromatherapy pain treatment devices and methods now present in the prior art, the present invention provides a new all natural aromatherapy thermal pad wherein the same can be utilized for providing convenience for the user when treating musculoskeletal aches and pains. The aromatherapy thermal pad comprises a first and second aroma-permeable outer layer, a padded inner layer, an herbal composition, and a securing means. Said first and second outer layers being secured along their edges. Housed within the first and second outer layers is a cotton padded inner layer for providing thermal retention and protection to an injured area from environmental hazards. The padded inner layer is affixed to the outer layers by stitching that dissects the inner space into chambers.

Chambers formed between the padded inner layer and an outer layer may be filled with an herbal composition. This herbal composition may consist of a variety of aromatic herbs and seeds. When the device is warmed the heat activates the natural aroma of the herbal composition thus providing aromatherapy benefits to user. When cooled the inner herbs freeze to provide an ice pack for swelling and pain relief without toxic gels or solutions. Such benefits are dependent upon the embodiment of the herbal composition element and may include promotion of relaxation, revitalization, or energizing of the body. The device may be heated in a microwave and then affixed to the affected area of a user by a securing means such as ties or closed loops. Once secured into place the device provides gentle warmth if heated or cooling relief if frozen, treating an injured area as well as offering aromatherapy benefits and using natural substances.

A method of using the aromatherapy thermal pad in a heated state is also contemplated. A user distributes the herbal composition evenly within the chambers of the device by shaking it. The device is then placed in a microwave, in either a wet or moistened state. A user then heats the pad for 30 to 60 seconds. After heating the aromatherapy pad is placed on the affected or injured area of a user and secured in place by the securing means. It then provides heat therapy to the affected area of a user.

It is therefore an object of the present invention to provide a new and improved all natural aromatherapy thermal pack device having all of the advantages of the prior art and none of the disadvantages.

Another object of the present invention is to provide a new and improved all natural aromatherapy thermal pack device having a cotton inner layer that reduces the risk of a user's skin burning by contact with the heated or frozen pad.

Still another object of the present invention is to provide a new and improved all natural aromatherapy thermal pad device that may be secured to a variety of body parts by cotton securing means.

A further object of the present invention is to provide a new and improved all natural aromatherapy thermal pad device having inner chambers filled with herbs and seeds that produce aromatherapy benefits upon warming of the device.

Another object of the present invention is to provide a method for treatment of musculoskeletal aches and pains using an all natural thermal pad filled with an aromatic herbal mixture.

Yet another object of the present invention is to provide a new and improved all natural aromatherapy heating pad device having durable and resilient construction that is simple and economically efficient to manufacture.

Other objects, features and advantages of the present invention will become apparent from the following detailed description taken in conjunction with the accompanying drawings.

### BRIEF DESCRIPTIONS OF THE DRAWINGS

The above invention will be better understood and the objects set forth above as well as other objects not stated above will become more apparent after a study of the following detailed description thereof. Such description makes use of the annexed drawings wherein:

FIG. 1 shows an aromatherapy thermal pad according to the present invention's preferred embodiment. The device has an elongated oval shape that is suited for use on a user's back, shoulders, arms, and legs.

FIG. 2 shoes an aromatherapy thermal pad according to the present invention and being shaped to conform to a user's kneed or elbow.

FIG. 3 shows an aromatherapy thermal pad according to the present invention in use on a user's lower back.

### DETAILED DESCRIPTION OF THE INVENTION

Reference is made herein to the attached drawings. Like reference numerals are used throughout the drawings to depict like or similar elements of the aromatherapy thermal pad device. For the purposes of presenting a brief and clear description of the present invention, the preferred embodiment will be discussed as used for treatment of pain. This is for representative purposes only and should not be considered limiting in any respect.

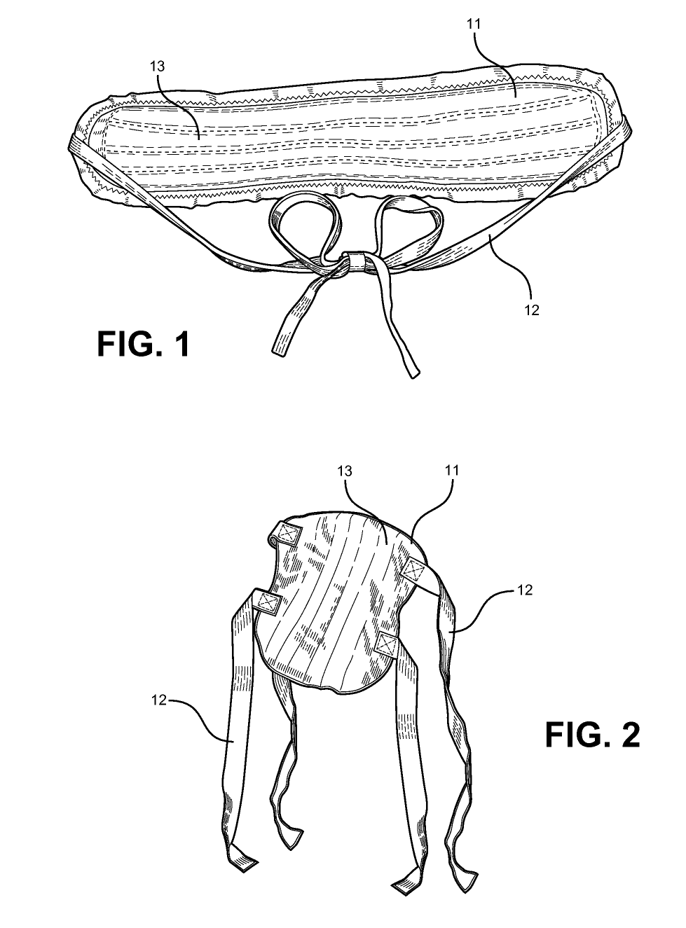

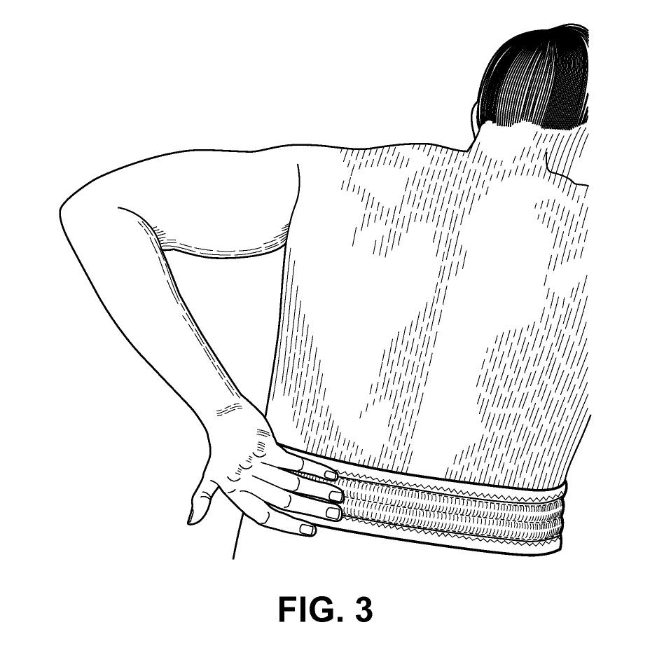

Referring now to FIG. 1, there is shown an all natural aromatherapy thermal pad according to the present invention. Said heating pad comprises a first and second layer forming an outer shell 11, a padded inner layer (not shown), a quantity of herbal composition (not shown), and securing means 12. The first and second outer layers are formed of an aroma-permeable soft cotton material. They are permanently secured by stitching along their edges to form an outer shell 11. Enclosed within said outer shell 11 is a padded inner layer. Said inner layer extends from each lateral seam of the outer shell to the opposing seam, filling the recess within the shell. The inner layer may be constructed of 100% cotton to promote an all natural product with low environmental impact. Use of a soft padded inner layer provides comfortable, cushioned protection to an injured area of a user, thus shielding an injury from environmental hazards and debris.

The outer shell 11 is affixed to the padded inner layer by rows of stitching that segments the inner enclosure into chambers 13. On at least one side of the device, said chambers of space are filled with a quantity of said herbal composition. The herbal composition comprises a mixture of aromatic herbs and seeds that may offer a variety of health benefits. The mixture may comprise lavender, basil, peppermint, chamomile, buckwheat, rice, and spearmint. Other herbal elements and combinations are contemplated as well. When the device is heated, the natural olfactory qualities of the herbal mixture are stimulated, enhancing their strength. The health benefits incurred are dependent on the herbal composition and may include improved relaxation, rejuvenation, energy, and physical sensations. Conversely, when the pack is placed in a freezer, the contents may freeze and retain the cold temperature for application onto a swelling area of the body.

The device is removably secured to a user by securing means disposed along laterally opposing ends of the shell. There may be a single pair of securing means or a plurality of the same as is necessary for the intended purpose of the device. The securing means may be all natural cotton ties that allow the device to be appropriately sized and tied about a user's injured area. Alternatively the securing means may be a closed loop of cotton that hooks to a wooden button. The device is made of a tea-dyed all natural fabric such as cotton. It may come in a variety of colors and shapes, with varying size and thickness of the cotton padded inner layer to provide proper heat transfer but reduce chances of skin burn or exposure.

Referring now to FIG. 2, there is shown an aromatherapy thermal pad adapted to conform to the shape of a user's knee or elbow. The device has a plurality of securing ties extending from opposing sides of the outer shell to allow it to be tied around both ends of a knee, elbow or similar joint. Shown in FIGS. 1 & 3 is an embodiment wherein the device is shaped like an extended oval that may be used on the back, shoulders, arms or legs. In another embodiment the device may be made without securing means for use as a shoe insert. In yet another embodiment the device may be formed into gloves having a securing means at the bottom edge of the device for securing to a user's wrists. Other structural forms are contemplated and the foregoing embodiments disclosed are not intended to be limiting. A primary feature is the use of all natural substances and ingredients for providing an aromatherapeutic device that may be used as a heat pack or cold pack, releasing a soothing and therapeutic aroma when heated.

A method of use is suggested wherein an individual shakes the thermal pad to evenly distribute the herbal composition contained within the inner chambers. The device is then placed in a microwave and heated for a period of time to allow heat absorption by the ingredients without overly heating the pad, which would then burn the patient. The exact amount of time is contemplated between 30-60 seconds in a standard microwave. This step may be executed while the thermal pad is dry or moist. A user then places the heated pad on the surface of the skin, adjacent to the injured area. The securing means are then used to removably affix the pad to the user thus relieving the user of the need to hold the therapy device in place. The device provides gentle comfortable warmth to an injury while also offering aromatic benefits.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim the following:

1) An aromatherapy thermal pad for treating injury, pain or swelling, comprising:
a first and second outer layer permanently secured together to form a shell enclosure with an internal volume,
said first and second layer being constructed of an aroma-permeable material;
an inner layer of padding attached to said first and second outer layer shell within said shell enclosure and affixed by stitching,
said stitching further dividing said internal volume into compartments;
a quantity of an herbal composition comprising herbs and seeds,
said herbal composition being distributed within said compartments.

2) The thermal pad of claim 1, further comprising:
a securing means fixedly attached to laterally opposing sides of said shell enclosure.

3) The thermal pad of claim 2, wherein:
said securing means comprises ties, that may be tied together around the body part of a user.

4) The thermal pad of claim 1, wherein:
said outer shell, said inner layer are constructed of 100% cotton material.

5) The thermal pad of claim 1, wherein:
said herbal composition comprising of a quantity of lavender, basil, spearmint, chamomile, buckwheat, rice, and spearmint.

6) A method of treating pain using an aromatherapy thermal pad comprising the steps of:
shaking an aromatherapy thermal pad gently to obtain even distribute of an internal herbal composition;
placing said thermal pad in a heating source;
heating said pad to a temperature acceptable for human skin contact without burn or pain;
removing said pad from a thermal source;
placing said thermal pad on the skin surface adjacent to an injured area of a user;
securing said thermal pad to an injured area of a user by a securing means.

7) The method of pain treatment of claim 6, further comprising the step of:
gently soaking said aromatherapy thermal pad prior to placing it in a heating source.
